(19) United States
(12) Patent Application Publication
(10) Pub. No.: US 2002/0183694 A1
(43) Pub. Date: Dec. 5, 2002

(54) Safe needle tube structure

(75) Inventor: Chiang-Li Lu, TaiChung (TW)

Correspondence Address: DOUGHERTY & TROXELL, 5205 LEESBURG PIKE, SUITE 1404, FALLS CHURCH, VA, 22041, US

(73) Assignee: Ming-Hsien Wang

(21) Appl. No.: 09/867,601

(22) Filed: May 31, 2001

(51) Int. Cl. A61M 5/00

(52) U.S. Cl. 604/187

(57) ABSTRACT

A safe needle tube structure is mainly comprised of an injection tube, a connection seat, and an injection handle. A blocking-and-supporting portion is pivotally arranged in the front end of the injection tube. A connection seat is arranged inside the injection tube. And, a connection portion is pivotally arranged in the front end of the connection seat. Two ring troughs are arranged oppositely in the front end of the connection portion. A fitting-in portion is in integration and in extension arranged in the tail end of the connection seat. A gliding trough is cut and arranged inside the fitting-in portion. And, an injection handle is also arranged through the interior of the gliding trough. A fitting-in block is in integration and in extension arranged in the front end of the injection handle. If the injection handle is pulled backward, then the blocking-and-supporting portion at the front end of the injection tube will make the injection needle stay at the front end of the injection tube and the injection tube may suck in medicine for injection. After the medicine is injected, the injection handle is rotated an angle to make the ring trough of the connection portion and the ring block of the injection needle be jointed to one body. The injection handle is moved backward to tail end. The fitting block of the injection handle is separated from the gliding trough of the connection seat. The injection needle stays inside the injection tube to reach the safe injection and recycling.

### FIELD OF THE INVENTION

The present invention relates to a safe needle tube structure, especially to a safe needle tube with relative safety and easy recycling during injection.

### BACKGROUND OF THE INVENTION

Accordingly, technology brings people with convenience in daily living, and also brings people a lots of troublesome problems, such as environment pollution, change of society custom and generation of new diseases, so technology is really a knife with two blades of advantage and disadvantage, and it all depends on how people use it. For the problem of environment pollution, in recent years, lots of manpower, money, and time are aggressively devoted to save the already damage-incurred environment. As for the problem of changing society custom, people greatly promote culture technology for hoping to change the society custom. As for the problem of new diseases, people then adopt passive and aggressive two sorts of doing. The passive doing is to control the bacteria within specific range and let it eliminate itself by time. The aggressive doing is total prevention and sterilization, such as spraying disinfectant, keeping environment clean, and categorization and recycling of medical instrument. And, the categorization and recycling of medical instrument is an emphasizing point of implementation. And, in the categorization and recycling of medical instrument, the injection needle is the easiest infection of disease. The prior injection needle is adopted with the method of fitting-in. After the injection tube is used, the injection needle is then set apart and categorized for recycling. But since the injection needle of this method easily pierces through the recycling plastic bag and punctures recycling personnel hurtfully, or due to the temporary negligence of medical personnel, so the medical personnel is incurred of puncturing and hurt, and furthermore the recycling personnel and medical personnel will be infected by diseases, so there are shortcomings in the usage of prior needle tube.

Because of so many shortcomings for above-mentioned prior injection needle tube, and according to many years'profession career on relative commercial manufacture of the present applicant and through continuous test and experiment, a safe needle tube structure with very practical usage of present invention has finally been created out.

### SUMMARY OF THE INVENTION

The main objective of the present invention is to make an injection handle be able to be separated from and connected with a connection seat by pivotally arranging with fitting-in blocks and fitting-in portions. And, the injection needle can stay inside the injection tube after usage to reach a safe function of injection needle tube structure.

Another objective of the present invention is to make connection seat unable to be separated from the injection tube due to too large pulling force by pivotally arranging with convex rings.

Another objective of the present invention is to make injection handle be able to only linearly move inside the injection tube by pivotally arranging with position-limited troughs and guiding portions. So, when the injection needle is moving, the injection needle will not be separated from the connection seat due to uneven applying force. And, the movement of the injection needle will not be interrupted and the injection of medicine will not be influenced.

Further, another objective of the present invention is to make the fitting-in block be able to be arranged through inside the gliding trough quickly by pivotally arranging with positioning blocks and positioning troughs.

### BRIEF DESCRIPTION OF THE DRAWINGS

FIG. 1 is a three-dimensional system drawing for the present invention.

FIG. 1A is a partly enlarging illustration for A part of FIG. 1.

FIG. 2 is a totally outer view drawing for the present invention.

FIG. 3 is a partly sectional view drawing for the after-assembly of the present invention.

FIG. 4 is an illustration for the before-connection of injection handle and connection seat of the present invention.

FIG. 5 is an illustration for the after-connection of injection handle and connection seat of the present invention.

FIG. 6 is a motion illustration for the before-movement of the injection handle of the present invention.

FIG. 7 is a motion illustration for the after-movement of the injection handle of the present invention.

FIG. 8 is a motion illustration for the separation of connection seat and injection needle of the present invention.

### DETAILED DESCRIPTION OF THE INVENTION

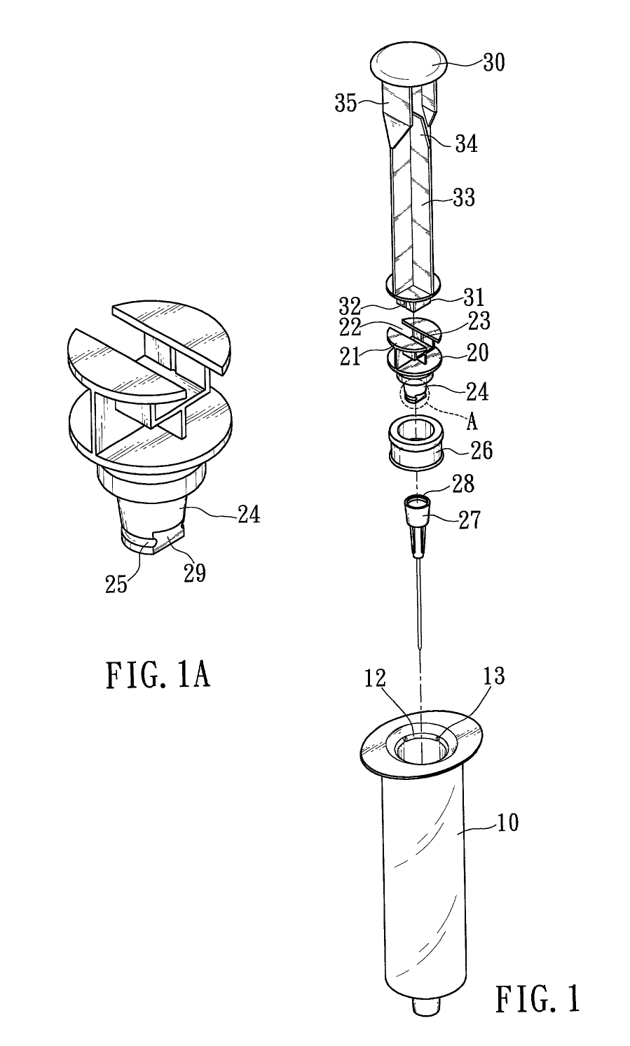

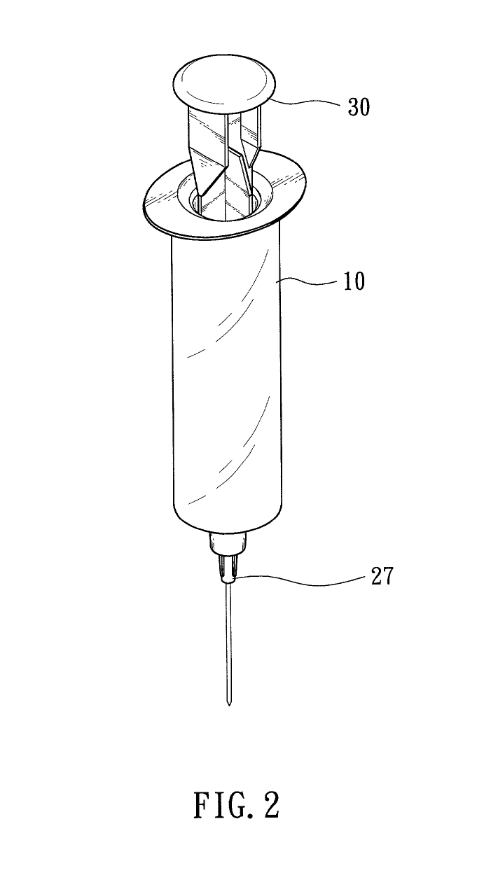

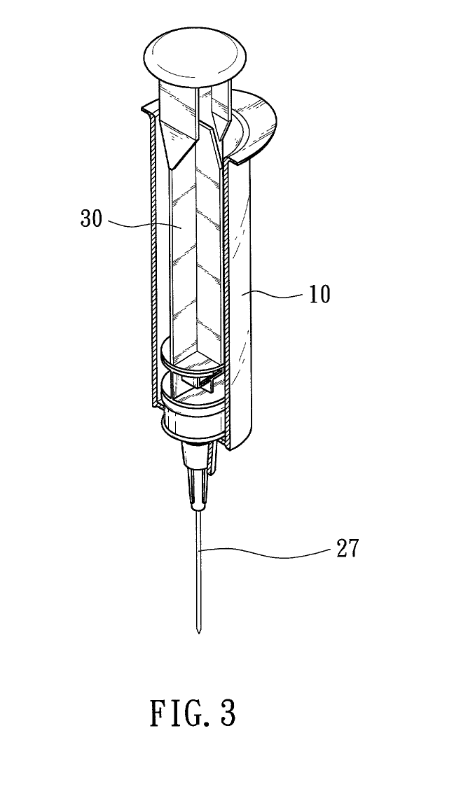

Please refer to FIG. 1 through FIG. 3. The present invention is comprised of the devices of an injection tube 10, a connection seat 20, and an injection handle 30 etc. Wherein the injection tube 10, the top end of which is pivotally arranged with a blocking-and-supporting portion 11, and the tail end of which is in integration and in extension arranged with a convex ring 12. And, four position-limited troughs 13, individually arranged on the convex ring 12, are set apart with specific angles. The connection seat 20, generally showing a shape of cone, the front end of which is in integration and in extension arranged with a connection portion 24, which is a column, and the front end of which is oppositely arranged with two ring troughs 25, and two ends of which is oppositely arranged with two cutting faces 29. And, the tail end of the connection seat is pivotally arranged with a fitting-in portion 21, the interior of which is cut and arranged with a gliding trough 22, and the interior of which two sides are oppositely arranged with two positioning blocks 23. And, then a ring-shaped washer is placed on the connection portion 24, and the front end of which is blocked and supported with a injection needle 27, and the tail end of which is oppositely arranged with two ring blocks 28 in the interior. The injection handle 30, the front end of which is in integration and in extension arranged with a fitting-in block 31, which shows as a rectangular block body, and two ends of which are oppositely arranged with two positioning troughs 32. And, the center of the injection handle 30 is in integration and in extension arranged four rib pieces 33, which are set apart with specific angles. And, a reverse angle is cut and arranged at the tail end of each rib pieces to become a guiding portion 34, and two cone piece bodies are oppositely arranged at the tail end of the injection handle 30 to become a gripping portion 35.

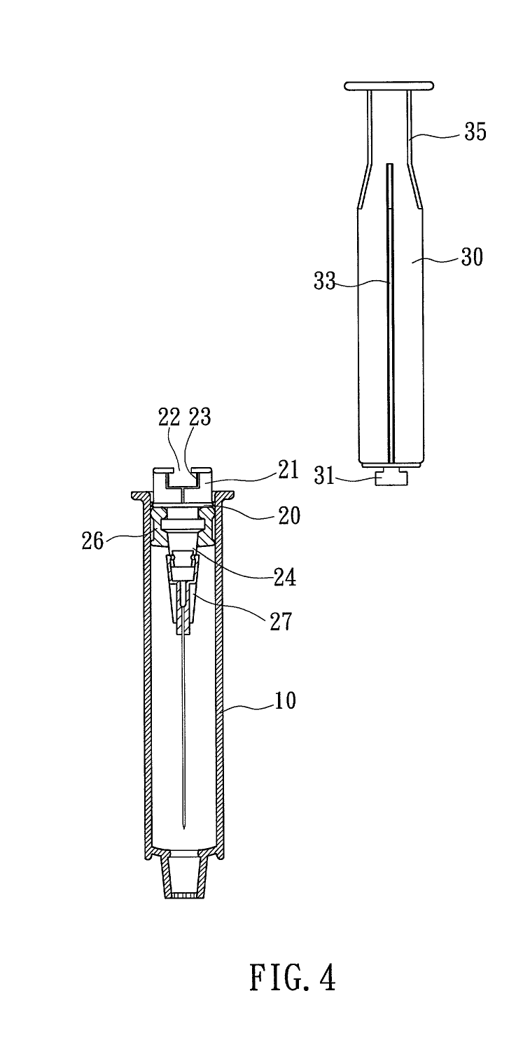

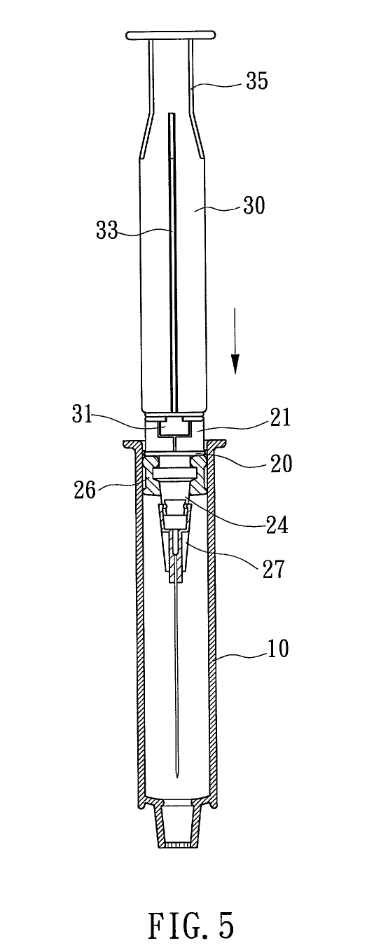

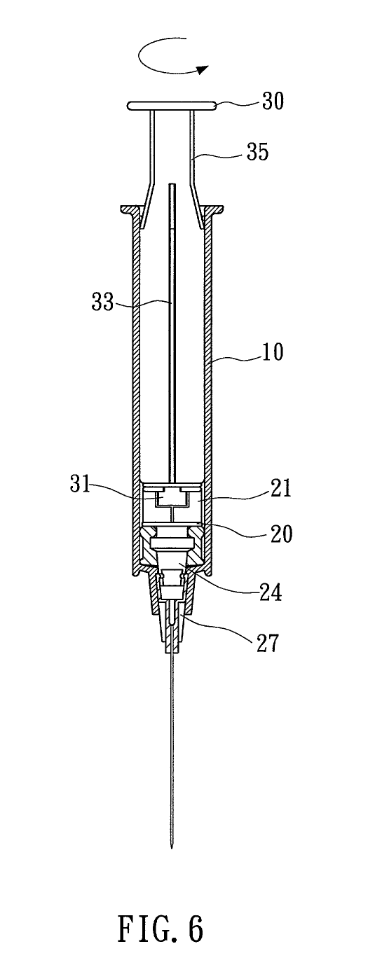

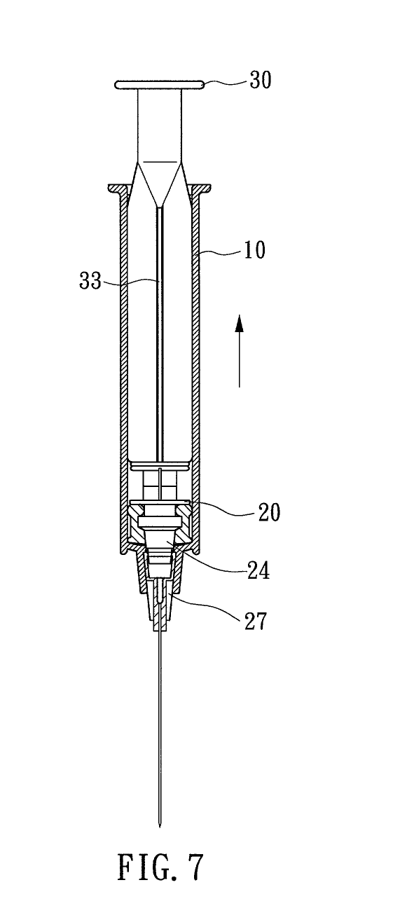

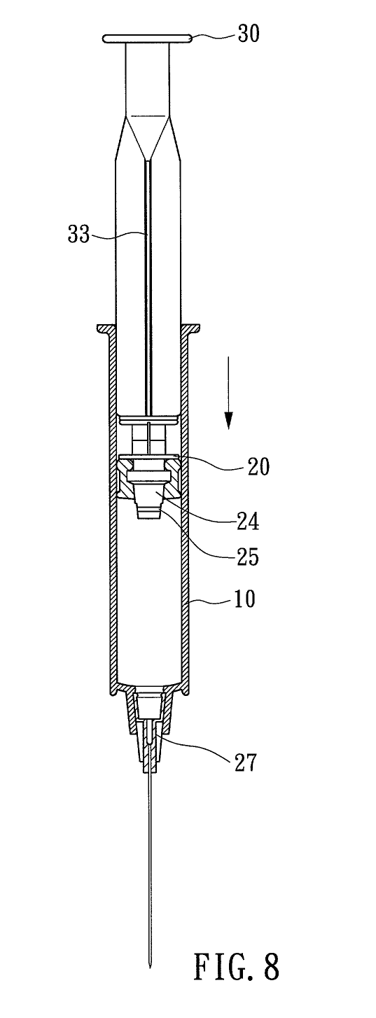

Please refer to FIG. 4 through FIG. 8. The connection seat 20 is placed through inside the injection tube 10. And, the injection handle 30 is placed through inside the gliding trough 22 by the fitting-in block 31 to make the injection handle 30 and the connection seat 20 be connected to one body. And, pushing against the injection handle 30 makes the connection seat 20 move forward. When the connection seat 20 moves to the front end of the injection needle 10, then the injection handle is rotated with a specific angle to separate the ring trough 25 of the connection portion at the front end of the connection seat 20 from the ring block 28 of the injection needle 27 and again to make the injection handle 30 be pulled backward. And, when the injection of medicine is over, the injection handle 30 is moved to the front end and is rotated with an angle. And, the ring trough 25 of the connection portion 24 and the ring block 28 of the injection 27 are connected to one body. And, the injection handle 30 is moved backward to the tail end of the injection tube 10 to make the fitting block 31 of the injection handle 30 be moved from the interior of the gliding trough 22. Then, the injection needle 27 stays inside the injection tube and reaches a safe function for an injection needle structure.

What is claimed is:

1. A safe needle tube structure, comprising:
an injection tube, the top end of which is pivotally arranged with a blocking-and-supporting portion, and the tail end of which is in integration and in extension arranged with a convex ring, and, four position-limited troughs, individually arranged on the convex ring, are set apart with specific angles;
a connection seat, the front end of which is in integration and in extension arranged with a connection portion, and the front end of which is oppositely arranged with two ring troughs, and, the tail end of the connection seat is pivotally arranged with a fitting-in portion, the interior of which is cut and arranged with a gliding trough, and the interior of which two sides are oppositely arranged with two positioning blocks, and, then a ring-shaped washer is placed on the connection portion, and the front end of which is blocked and supported with a injection needle; and
an injection handle, the front end of which is in integration and in extension arranged with a fitting-in block, which shows as a rectangular block body, and two ends of which are oppositely arranged with two positioning troughs, and, the center of the injection handle is in integration and in extension arranged four rib pieces, which are set apart with specific angles, and, a reverse angle is cut and arranged at the tail end of each rib pieces to become a guiding portion, and two cone piece bodies are oppositely arranged at the tail end of the injection handle to become a gripping portion.
the connection seat is placed through inside the injection tube. And, the injection handle is placed through inside the gliding trough by the fitting-in block to make the injection handle and the connection seat be connected to one body. And, pushing against the injection handle makes the connection seat move forward. When the connection seat moves to the front end of the injection needle, then the injection handle is rotated with a specific angle to separate the ring trough of the connection portion at the front end of the connection seat from the ring block of the injection needle. And, when the injection of medicine is over, the injection handle is moved to the front end and is rotated with an angle. And, the connection portion of the connection seat and the injection needle are connected to one body. And, the injection handle is moved backward to the tail end of the injection tube to make the fitting block of the injection handle be moved from the interior of the gliding trough. Then, the injection needle stays inside the injection tube and reaches relative safety.
